(19) United States
(12) Patent Application Publication
(10) Pub. No.: US 2022/0184164 A1
(43) Pub. Date: Jun. 16, 2022

(54) PREPARATIONS CONTAINING BERRY EXTRACTS FOR USE IN THE PROPHYLAXIS AND/OR TREATMENT OF VIRAL INFECTIONS CAUSED BY HERPESVIRIDAE

(71) Applicant: Evonik Operations GmbH, Essen (DE)

(72) Inventors: Jean-Luc HERBEAUX, Cascais (PT); Norbert WINDHAB, Hofheim (DE); Christoph BRÜCHER, Eschborn (DE); Anne BENEDIKT, Frankfurt (DE); Maria STEINKE, Hoechberg (DE); Jochen BODEM, Geroldshausen (DE)

(73) Assignee: Evonik Operations GmbH, Essen (DE)

(21) Appl. No.: 17/598,647

(22) PCT Filed: Mar. 27, 2020
(86) PCT No.: PCT/EP2020/058651

§ 371 Date: Sep. 27, 2021

(30) Foreign Application Priority Data
Mar. 29, 2019 (EP) 19166083.6

(51) Int. Cl. A61K 36/45; A61K 36/185; A61K 31/352; A61P 31/22; A61K 45/06

(52) U.S. Cl. CPC A61K 36/45; A61K 36/185; A61K 45/06; A61P 31/22; A61K 31/352

(57) ABSTRACT

A composition may be used in treating or preventing a virus infection in a subject, wherein the virus is from the Herpesviridae family and wherein the composition includes an extract of black currants and bilberries. The composition may contain an extract from black currants and bilberries in a weight ratio of 0.5:1 to 1:0.5

The present invention is related to composition for use in treating or preventing a virus infection in a subject, wherein the virus is from the Herpesviridae family and wherein the composition comprises an extract of black currants and bilberries.

Anthocyanins are water-soluble vacuolar pigments that may appear red, purple or blue, depending on the surrounding pH-value. Anthocyanins belong to the class of flavonoids, which are synthesized via the phenylpropanoid pathway. They occur in all tissues of higher plants, mostly in flowers and fruits and are derived from anthocyanidins by addition of sugars. Anthocyanins are glycosides of flavylium salts. Each anthocyanin thus comprises three component parts: the hydroxylated core (the aglycone); the saccharide unit; and the counterion. Anthocyanins are naturally occurring pigments present in many flowers and fruit and individual anthocyanins are available commercially as the chloride salts, e.g. from Polyphenols Laboratories AS, Sandnes,

Norway. The most frequently occurring anthocyanins in nature are the glycosides of cyanidin, delphinidin, malvidin, pelargonidin, peonidin and petunidin.

It is known that anthocyanins, especially resulting from fruit intake, have a wide range of biological activities, including antioxidant, anti-inflammatory, antimicrobial and anti-carcinogenic activities, improvement of vision, induction of apoptosis, and neuroprotective effects. Particularly suitable fruit sources for the anthocyanins are cherries, bilberries, blueberries, black currants, red currants, grapes, cranberries, strawberries, and apples and vegetables such as red cabbage. Bilberries, in particular Vaccinium myrtillus, and black currants, in particular Ribes nigrum, are especially suitable.

Bilberries contain diverse anthocyanins, including delphinidin and cyanidin glycosides and include several closely related species of the genus Vaccinium, including Vaccinium myrtillus (bilberry), Vaccinium uliginosum (bog bilberry, bog blueberry, bog whortleberry, bog huckleberry, northern bilberry, ground hurts), Vaccinium caespitosum (dwarf bilberry), Vaccinium deliciosum (Cascade bilberry), Vaccinium membranaceum (mountain bilberry, black mountain huckleberry, black huckleberry, twin-leaved huckleberry), Vaccinium ovalifolium (oval-leafed blueberry, oval-leaved bilberry, mountain blueberry, high-bush blueberry).

Dry bilberry fruits of V. myrtillus contain up to 10% of catechin-type tannins, proanthocyanidins, and anthocyanins. The anthocyanins are mainly glucosides, galactosides, or arabinosides of delphinidin, cyanidin, and—to a lesser extent—malvidin, peonidin, and petunidin (cyanidin-3-O-glucoside (C3G), delphinidin-3-O-glucoside (D3G), malvidin-3-O-glucoside (M3G), peonidin-3-O-glucoside and petunidin-3-O-glucoside). Flavonols include quercetin- and kaempferol-glucosides. The fruits also contain other phenolic compounds (e.g., chlorogenic acid, caffeic acid, o-, m-, and p-coumaric acids, and ferulic acid), citric and malic acids, and volatile compounds.

Black currant fruits (R. nigrum) contain high levels of polyphenols, especially anthocyanins, phenolic acid derivatives (both hydroxybenzoic and hydroxycinnamic acids), flavonols (glycosides of myricetin, quercetin, kaempferol, and isorhamnetin), and proanthocyanidins (between 120 and 166 mg/100 g fresh berries). The main anthocyanins are delphinidin-3-O-rutinoside (D3R) and cyanidin-3-O-rutinoside (C3R), but delphinidin- and cyanidin-3-O-glucoside are also found (Gafner, Bilberry—Laboratory Guidance Document 2015, Botanical Adulterants Program).

EP 1443948 A1 relates to a process for preparing a nutritional supplement (nutraceutical) comprising a mixture of anthocyanins from an extract of black currants and bilberries. Anthocyanins were extracted from cakes of fruit skin produced as the waste product in fruit juice pressing from V. myrtillus and R. nigrum. It could be shown that the beneficial effects of individual anthocyanins are enhanced if instead of an individual anthocyanin, a combination of different anthocyanins is administered orally, in particular a combination comprising both mono and disaccharide anthocyanins. It is thought that the synergistic effect arises at least in part from the different solubilities and different uptake profiles of the different anthocyanins.

Herpesviridae is a large family of DNA viruses that cause infections and certain diseases in humans such as oral herpes, chicken pox and infectious mononucleosis-like syndrome. Additionally, they can be connected to serious pathophysiology including Alzheimer's disease, Burkitt's lymphoma and Kaposi's sarcoma. Latent, recurring infections are also typical of this group of viruses, e.g. over 50% of the population worldwide is seropositive for human cytomegalovirus (hCMV). This ubiquitous herpes virus is the cause of widespread infections in humans and, although benign in immunocompetent hosts, patients with immature or compromised immune systems (as AIDS patients or organ transplant recipients) suffer from life-threatening complications.

In total more than 130 herpesviruses are known, however nine herpesvirus types are known to cause disease in humans, such as herpes simplex viruses 1 and 2 (HSV-1 and HSV-2, also known as HHV1 and HHV2) causing oral and/or genital herpes, as well as other herpes simplex infections, targeting mucoepithelial cells and neuronal latency. The varicella-zoster virus (VZV, HHV-3) is also targeting mucoepithelial cells (neuronal latency) and causes chickenpox and shingles. Epstein—Barr virus (EBV, HHV-4) is targeting B cells (including latency in B cells) and epithelial cells and is the cause of Infectious mononucleosis, Burkitt's lymphoma, CNS lymphoma in AIDS patients, post-transplant lymphoproliferative syndrome (PTLD), nasopharyngeal carcinoma and HIV-associated hairy leukoplakia. The human cytomegalovirus (HCMV, HHV-5) is targeting monocytes and epithelial cells (monocytes as site of latency) and causes infectious mononucleosis-like syndrome and retinitis. Human herpesvirus 6A and 6B (HHV-6A and HHV-6B) targets T cells (including site of latency) and causes sixth disease (roseola infantum or exanthem subitum). Human herpesvirus 7 (HHV-7) targets T cells as well and is the cause of drug-induced hypersensitivity syndrome, encephalopathy, hemiconvulsion-hemiplegia-epilepsy syndrome, hepatitis infection, post infectious myeloradiculoneuropathy, pityriasis rosea, and the reactivation of HHV-4, leading to “mononucleosis-like illness”. The Kaposi's sarcoma-associated herpesvirus (KSHV, HHV-8) is targeting lymphocytes and other cells and causes Kaposi's sarcoma, primary effusion lymphoma, some types of multicentric Castleman's disease.

Herpesviruses are known for their ability to establish lifelong infections in the host, which is achieved through immune evasion. Interestingly, herpesviruses have many different ways of evading the immune system, such as mimicking human interleukin 10 (hIL-10) or downregulation of the major histocompatibility complex II (MHC II) in infected cells.

During the past decade a better understanding of the replication and disease-causing state of herpes viruses has been achieved in part due to the development of potent antiviral compounds that target these viruses. While some of these antiviral therapies are considered safe and efficacious (acyclovir, penciclovir), some have toxicities associated with them (ganciclovir and foscarnet). The most serious side effect of acyclovir is neurotoxicity, which usually occurs in subjects with compromised renal function who attain high serum concentrations of drug (Revankar et al., 1995). Neurotoxicity is manifest as lethargy, confusion, hallucinations, tremors, myoclonus, seizures, extrapyramidal signs, and changes in state of consciousness, developing within the first few days of initiating therapy. These signs and symptoms usually resolve spontaneously within several days of discontinuing acyclovir. Resistance of HSV to acyclovir has become an important clinical problem, especially among immunocompromised patients exposed to long-term therapy (Englund et al., 1990).

In the context it was surprisingly found, that an extract of black currants and bilberries, mediates strong inhibition of herpes virus infection and replication, and there is a surprising synergistic effect between the extract of black currants and the extract of bilberries. Thus, the present invention is based on the use of extracts of black currants and bilberries as anti-viral agent in the treatment and prophylaxis of herpes infection. Therefore, extracts of black currants and bilberries could be an important solution for a variety of herpes infections as well as their related diseases by combining the antiviral effect with its positive influence on cell viability and no toxicity.

The present invention is related to a composition for use in treating or preventing a virus infection in a subject, wherein the virus is from the Herpesviridae family and wherein the composition comprises an extract of black currants and bilberries.

In one embodiment the composition is for use in treating or preventing a virus infection, wherein the virus is from the sub-family Alphaherpesvirinae or Gammaherpesvirinae, preferably wherein the subject is human.

In another embodiment the composition according to the present invention is especially for use in treating or preventing a virus infection in a human host, the virus being selected from herpes simplex viruses 1 and 2 (HSV-1 and HSV-2, HHV1 and HHV2),
varicella-zoster virus (VZV, HHV-3),
Epstein—Barr virus (EBV, HHV-4),
human cytomegalovirus (HCMV, HHV-5),
human herpesvirus 6A and 6B (HHV-6A and HHV-6B),
human herpesvirus 7 (HHV-7), and
Kaposi's sarcoma-associated herpesvirus (KSHV, HHV-8).

The virus is preferably HSV-1, EBV, CMV or HHV-8, more preferably HSV-1, mCMV and HHV-8 and the composition preferably suppresses viral infection.

Moreover, herpesviruses represent the most frequently detected pathogens in the brain. Under constant immune pressure, these infections are largely asymptomatic in healthy hosts. However, many neurotropic herpesviruses have been directly connected with central nervous system pathology in the context of other stressors and genetic risk factors. There are indications that neurotropic herpesviruses, such as herpes simplex virus 1 (HSV-1) and human herpesvirus 6 (HHV-6) contribute to neurodegenerative disease pathology, such as Alzheimer's disease (AD) (Hogestyn et al., Neural Regeneration Research 13 (2), 211-221, 2018). For example, the herpes simplex virus HSV-1 has been found in the same areas as amyloid plaques. It has been shown that HSV-1 induces AD-related pathophysiology and pathology, including neuronal production and accumulation of amyloid beta (A13), hyperphosphorylation of tau proteins, dysregulation of calcium homeostasis, and impaired autophagy (Harris & Harris Frontiers in Aging Neuroscience Vol 10 (48), 2018). This suggested the possibility that AD could be treated or prevented with antiviral medication.

It is further also preferred to use the composition according to the present invention for treating or preventing a virus infection with Ateline herpesvirus 1 (spider monkey herpesvirus), Bovine herpesvirus 2 (which causes bovine mammillitis and pseudo-lumpyskin disease), Cercopithecine herpesvirus 1 (also known as Herpes B virus, causes a herpes simplex-like disease in macaques, usually fatal if symptomatic and untreated in humans), Macacine herpesvirus 1, Bovine herpesvirus 1 (causes infectious bovine rhinotracheitis, vaginitis, balanoposthitis, and abortion in cattle), Bovine herpesvirus 5 (causes encephalitis in cattle), Bubaline herpesvirus 1, Caprine herpesvirus 1 (causes conjunctivitis and respiratory disease in goats), Canine herpesvirus 1 (causes a severe hemorrhagic disease in puppies), Equine herpesvirus 1 (causes respiratory disease, neurological disease/paralysis, and spontaneous abortion in horses), Equine herpesvirus 3 (causes coital exanthema in horses), Equine herpesvirus 4 (causes rhinopneumonitis in horses), Equine herpesvirus 8, Equine herpesvirus 9, Feline herpesvirus 1 (causes feline viral rhinotracheitis and keratitis in cats), Suid herpesvirus 1 (causes Aujeszky's disease, also called pseudorabies),

Anatid herpesvirus 1, Columbiform herpesvirus 1, Gallid herpesvirus 2 (causes Marek's disease), Gallid herpesvirus 3 (GaHV-3 or MDV-2), Meleagrid herpesvirus 1 (HVT), Peacock herpesvirus 1 Gallid herpesvirus 1 (causes infectious laryngotracheitis in birds), Psittacid herpesvirus 1 (causes Pacheco's disease in birds),

Porcine herpesvirus 2 (causes inclusion body rhinitis in swine),

Alcelaphine herpesvirus 1 (causes bovine malignant catarrhal fever), Alcelaphine herpesvirus 2 (causes an antelope and hartebeest version of MCF), Ateline herpesvirus 2, Bovine herpesvirus 4, Cercopithecine herpesvirus 17, Equine herpesvirus 2 (causes equine cytomegalovirus infection), Equine herpesvirus 5, Equine herpesvirus 7, Japanese macaque rhadinovirus, Leporid herpesvirus 1, Murid herpesvirus 4 (Murine gammaherpesvirus-68, MHV-68), Cyprinid herpesviruses 1, 2 and 3 (CyHV1, CyHV2 and CyHV3) causing disease in common carp, goldfish and koi respectively.

In a preferred embodiment, the black currants are the fruit of Ribes nigrum and/or the bilberries are the fruit of Vaccinium myrtillus. It is further preferred, when the composition contains an extract from black currants and bilberries in a weight ratio of 0.5:1 to 1:0.5. In an advantageous configuration of the present invention, the composition is an extract of the pomaces from black currants and bilberries.

It is particularly preferred, when the composition comprises anthocyanins and the anthocyanins are present in the composition at a concentration of at least 25 weight-%, preferably at least 30 weight-%, or at least 35 weight-%, or at least 40 weight-%, or at least 45 weight-%, or at least 50 weight-%.

It is preferred, according to the present invention, when the extract is an alcoholic extract, preferably a methanol extract. The extract is preferably produced by a process comprising the steps of
extraction of black currants and/or bilberries,
purification via chromatography,
mixing of the extract(s) with water and
spray-drying of the mixture.

One example of such a process is disclosed in EP1443948.

In a preferred embodiment, maltodextrin is added to the composition.

The composition according to the present invention preferably contains at least three monosaccharide anthocyanins. Moreover, it preferably contains at least one monosaccharide anthocyanin in which the saccharide is arabinose or at least one disaccharide anthocyanin in which the disaccharide is rutinose. The composition preferably contains anthocyanins with at least two different aglycones, more preferably at least four. Especially preferably the composition contains anthocyanins in which the aglycone units are cyanidin, peonidin, delphinidin, petunidin, malvidin and optionally also pelargonidin. In one preferred embodiment, the composition also contains at least one trisaccharide anthocyanin. The disaccharide anthocyanins are more water-soluble than the monosaccharides; moreover, cyanidin and delphinidin anthocyanins are amongst the most water-soluble anthocyanins.

In an advantageous embodiment of the present invention anthocyanins are selected from cyanidin-3-glucoside, cyanidin-3-galactoside, cyanidin-3-arabinoside, delphinidin-3-glucoside, delphinidin-3-galactoside, delphinidin -3-arabinoside, petunidin-3-glucoside, petunidin-3-galactoside, petunidin-3-arabinose, peonidin-3-glucoside, peonidin-3-galactoside, peonidin-3-arabinose, malvidin-3-glucoside, malvidin-3-galactoside, malvidin-3-arabinose, cyanidin-3-rutinoside, delphinidin-3-rutinoside. The anthocyanins are preferably selected from cyanidin-3-glucoside, cyanidin-3-rutinoside, delphinidin-3-glucoside, delphinidin-3-rutinoside, cyanidin-3-galactoside, delphinidin-3-galactoside.

The anthocyanins can be from natural sources or from synthetic productions. Natural sources are preferably selected from fruits, flowers, leaves, stems and roots, preferably violet petal, seed coat of black soybean. Preferably anthocyanins are extracted from fruits selected from: açai, black currant, aronia, eggplant, blood orange, marion blackberry, black raspberry, raspberry, wild blueberry, cherry, queen Garnet plum, red currant, purple corn (Z. mays L.), concord grape, norton grape, muscadine grape, red cabbage, okinawan sweet potato, Ube, black rice, red onion, black carrot. Particularly suitable fruit sources for the anthocyanins are cherries, bilberries, blueberries, black currants, red currants, grapes, cranberries, strawberries, black chokeberry, and apples and vegetables such as red cabbage. Bilberries, in particular Vaccinium myrtillus, and black currants, in particular Ribes nigrum, are especially suitable. It is further preferred to use plants enriched with one or more of anthocyanins as natural sources, preferably plants enriched with delphinidin-3-rutinoside.

The counterion in the anthocyanins in the composition of the invention may be any physiologically tolerable counter anions, e.g. chloride, succinate, fumarate, malate, maleate, citrate, ascorbate, aspartate, glutamate, etc. Preferably however the counterion is a fruit acid anion, in particular citrate, as this results in the products having a particularly pleasant taste. Besides the anthocyanins, the composition may desirably contain further beneficial or inactive ingredients, such as vitamins (preferably vitamin C), flavones, isoflavones, anticoagulants (e.g. maltodextrin, silica, etc.), desiccants, etc.

It is preferred when the composition comprises anthocyanins and is to be administered to the subject in a dose of the anthocyanins/regimen of 1 to 10 oral dosages of at least 80 mg anthocyanins each per day, preferably 3 to 6 oral dosages of at least 80 mg anthocyanins each per day.

It is known that viral infections can occur when a medical device is used on a subject. This is particularly the case when the device, such as a catheter or feeding tube, is to be retained in the subject for any length of time, e.g. the dwell time of the device in the subject is more than 24 hours.

Accordingly, in a preferred embodiment, the composition is for use with a medical device which is to be inserted into the subject, or wherein the subject has had a medical device inserted, optionally wherein the inserted device is transdermal or endotracheal. In a preferred embodiment, the composition is to be administered at a site of insertion of the medical device into the subject. It is further preferred, when the medical device is for endotracheal intubation, or parenteral nutrition.

In a specific configuration, the medical device is a needle, a catheter, a port, an intubation device or tube, a nebulizer, an implant, a vascular access catheter, a brain microcatheter, a peripherally inserted central catheter, a chronic central venous catheter, an implanted port, an acute central venous catheter, a midline catheter, a short peripheral intravenous catheter, or a dialysis catheter.

It is preferred, when a dwell time of the medical device in the subject is more than 24 hours, more than 48 hours, more than 72 hours, more than one week, more than 2 weeks, more than 3 weeks, preferably wherein the dwell time is more one week, more than 2 weeks or more than 3 weeks.

In a further advantageous configuration, the composition is to be administered to the subject as parenteral bolus injection or infusion or parenteral nutritional solution. It is also preferred to use the composition to stabilize critical patients, where lifesaving treatments are not effective, and no last-line treatment is available (due to lack of treatment options).

The composition according to the present invention is to be administered to the subject, reaching a concentration in the target compartment at least 30 μg/ml, preferably at least 100 μg/ml. Target compartment are blood and lymph, specifically the medium surrounding the cells of the immune system, which are infected by the Herpesviridae, preferably Peripheral Blood Mononuclear Cells(PBMCs), especially B cells, T cells, dendritic cells.

In a preferred embodiment, the subject is a human, preferably the subject is pregnant or immunocompromised or taking an immunosuppressant or is a carrier of a virus from the Herpesviridae family, preferably wherein the subject is a carrier of herpes simplex virus, Epstein—Barr or human cytomegalovirus.

In another embodiment, the subject is infected with Kaposi's sarcoma-associated herpesvirus (KSHV, HHV-8), optionally wherein the subject is HIV-positive or is suffering from AIDS.

In a preferred embodiment, the virus infection is in the liver or kidney. The tested berry extracts show a broad activity in contrast to known antivirals. Therefore, it can be for use, when a liver infection is diagnosed (EBV, CMV or HSV). Since the berry extracts shall not be toxic to kidney, it could also be used after transplantation as a prophylaxis.

Another aspect of the present invention is related to a composition for use for the prevention or treatment of a cancer associated with a virus from the Herpesviridae family, wherein the composition comprises an extract of black currants and bilberries, optionally wherein:

(i) the virus is EBV and the cancer is lymphoma (including Hodgkin lymphoma and Burkitts lymphoma), nasopharyngeal cancer, gastric cancer, or breast cancer; or

(ii) the virus is HHV-8 and the cancer is Kaposi's sarcoma, primary effusion lymphoma, HHV-8-associated multicentric Castleman disease, or breast cancer.

Another aspect of the present invention is related to a composition for the prevention or treatment of an autoimmune disease associated with a virus from the Herpesviridae family, wherein the composition comprises an extract of black currants and bilberries, optionally wherein:

(i) the virus is EBV and the autoimmune disease is systemic lupus erythematosus (SLE), rheumatoid arthritis (RA), Sjogren's syndrome or multiple sclerosis; or

(ii) the virus is HSV-1 and the autoimmune disease is multiple sclerosis.

In these aspects the components of the composition comprising the extract of black currants and bilberries may be as described above.

The composition for use according to the present invention is preferably useful for subjects exposed to physical or emotional stress, or subject is suffering from fatigue, depression or anxiety, which may lead to reactivation of latent herpesvirus infections.

Moreover, the composition is useful for the prevention or treatment of Alzheimer disease. In particular, as shown in the Examples the inventors have found that a composition comprising an extract of black currants and bilberries surprisingly reduces virus related β-amyloid deposition.

Therefore, another aspect of the invention covers a composition for use for the prevention or treatment of Alzheimer disease, wherein the composition comprises an extract of black currants and bilberries, wherein the composition reduces β-amyloid plaque formation, optionally wherein the composition reduces β-amyloid plaque formation by reducing or preventing a virus infection.

The reduction of viral infection may be assessed by performing PCR on a blood sample to determine reduction in viral copy number, the viral copy number can be used to determine whether the infection is passive or active. The composition can be used both to prevent viral infection and to prevent viral reactivation.

In a specific configuration, the composition for use for the prevention or treatment of Alzheimer disease reduces brain tissue inflammation. An encephalitis may also be prevented in this context.

In this aspect the components of the composition comprising the extract of black currants and bilberries may be as described above.

A further aspect of the present invention is a topical composition comprising an extract of black currants and bilberries, wherein the composition further comprises a pharmaceutically acceptable excipient suitable for a topical composition that is to be administered to the skin, preferably wherein the pharmaceutically acceptable excipient comprises one or more of a tonicity adjusting agent, a buffering agent, a preservative, an antioxidant, a stabilizer, a pH adjusting agent, a penetration enhancer, a surfactant and a humectant. It is further preferred, when the topical composition is a lip balm or lip protection product.

A further aspect of the present invention is an eye drop composition comprising an extract of black currants and bilberries, wherein the composition further comprises a pharmaceutically acceptable excipient suitable for a composition that is to be administered to the eye, preferably wherein the pharmaceutically acceptable excipient comprises one or more of a tonicity adjusting agent, a buffering agent, a preservative, an antioxidant, a stabilizer, a pH adjusting agent, a penetration enhancer, a surfactant and a humectant.

The present invention also refers to
a composition comprising an analgesic and an extract of black currants and bilberries, preferably wherein the analgesic is ibuprofen or paracetamol/acetaminophen,
a composition for use in treating pain associated with a virus infection in a subject, wherein the virus is from the Herpesviridae family,
a combined preparation comprising an analgesic, and an extract of black currants and bilberries, for simultaneous, separate or sequential use in medicine,
a topical composition comprising an analgesic, and an extract of black currants and bilberries,
a composition comprising an antiviral agent, and an extract of black currants and bilberries, optionally wherein the antiviral agent is acyclovir, ganciclovir, valganciclovir, foscarnet, famciclovir, penciclovir, valaciclovir, or letermovir,
a composition which is in the form of a topical composition or eye drops, preferably wherein the antiviral agent is acyclovir,
a combined preparation comprising an antiviral agent, and an extract of black currants and bilberries, for simultaneous, separate or sequential use in medicine.

A combined preparation is one which comprises separately packaged active components which are to be combined in use, i.e. by being administered simultaneously, separately or sequentially to the subject.

Analgesic compounds are preferably selected from acetylsalicylic acid, Diclofenac, Dexibuprofen, Dexketoprofen, Flurbiprofen, Ibuprofen, Indometacin, Ketoprofen, Meloxicam, Nabumeton, Naproxen, Phenylbutazon, Piroxicam, Phenazon, Propyphenazon, rofecoxib, Celecoxib, Etoricoxib, Parecoxib, Metamizol, Paracetamol/Acetaminophen.

The antiviral agent indicated above is preferably a Herpesviridae antiviral agent. By Herpesviridae antiviral agent is meant an agent that can be used to treat or prevent an infection by a virus from the Herpesviridae family, and can itself be active against the virus or can be a prodrug that is metabolized in the body to an active agent. An example of the latter is valganciclovir, which is a prodrug of ganciclovir. Preferably the Herpesviridae antiviral agent is an inhibitor of DNA replication, optionally a DNA polymerase inhibitor or a DNA terminase complex inhibitor. In particular, the DNA polymerase inhibitor may be a nucleoside analogue or a pyrophosphate analogue. In a preferred embodiment the antiviral agent is acyclovir, ganciclovir, valganciclovir, foscarnet, famciclovir, penciclovir, valaciclovir, or letermovir.

For all the compositions described above it is advantageous, when the black currants are the fruit of Ribes nigrum and/or the bilberries are the fruit of Vaccinium myrtillus. It is further preferred, when the composition contains an extract from black currants and bilberries in a weight ratio of 0.5:1 to 1:0.5. In an advantageous configuration of the present invention, the composition is an extract of the pomaces from black currants and bilberries. It is particularly preferred, when the composition comprises anthocyanins and the anthocyanins are present in the composition at a concentration of at least 25 weight-%, preferably at least 30 weight-%, or at least 35 weight-%, or at least 40 weight-%, or at least 45 weight-%, or at least 50 weight-%. It is preferred, according to the present invention, when the extract is an alcoholic extract, preferably a methanol extract.

The present invention is also related to an agent with antiviral activity for treating or preventing a virus infection in a subject, wherein the virus is from the Herpesviridae family with a level of efficacy of 2 log levels, and an antiviral agent which is non-toxic.

The invention is also referring to an agent with antiviral activity for treating or preventing a virus infection in a subject, wherein the virus is from the Herpesviridae family with a level of efficacy of 2 log levels, which is not killing more than 30%, preferably not more than 20%, more preferably not more than 10% of cells in a cell-based assay in mammalian cells, preferably BHK cells.

This agent with antiviral activity preferably comprises one or more anthocyanins selected from cyanidin-3-glucoside, cyanidin-3-galactoside, cyanidin-3-arabinoside, delphinidin-3-glucoside, delphinidin-3-galactoside, delphinidin -3-arabinoside, petunidin-3-glucoside, petunidin-3-galactoside, petunidin-3-arabinose, peonidin-3-glucoside, peonidin-3-galactoside, peonidin-3-arabinose, malvidin-3-glucoside, malvidin-3-galactoside, malvidin-3-arabinose, cyanidin-3-rutinoside, delphinidin-3-rutinoside. The anthocyanins are preferably selected from cyanidin-3-glucoside, cyanidin-3-rutinoside, delphinidin-3-glucoside, delphinidin-3-rutinoside, cyanidin-3-galactoside, delphinidin-3-galactoside.

As noted above, the present invention is also related to a composition for use, wherein the composition is for use with a medical device which is to be inserted into the subject, or wherein the subject has had a medical device inserted, optionally wherein the device is inserted via the nose or mouth. It is preferred, when the medical device is a needle, a catheter, a port, an intubation device or tube, or a nebulizer. It is further preferred, when a dwell time of the medical device in the subject is more than 24 hours, more than 48 hours, more than 72 hours, more than one week, more than 2 weeks, more than 3 weeks, preferably wherein the dwell time is more than one week, more than 2 weeks or more than 3 weeks.

The invention further refers to a medical device suitable for insertion into a subject, the medical device comprising a coating composition on an exterior surface of the device, wherein the coating composition comprising an extract of black currants and bilberries. It is preferred, when the medical device is a needle, a catheter, an intubation device or tube, or a nebulizer, preferably wherein the exterior surface of the medical device is plastic.

It is further preferred, when the black currants are the fruit of Ribes nigrum and/or the bilberries are the fruit of Vaccinium myrtillus. It is further preferred, when the composition contains an extract from black currants and bilberries in a weight ratio of 0.5:1 to 1:0.5. In an advantageous configuration of the present invention, the composition is an extract of the pomaces from black currants and bilberries. It is particularly preferred, when the composition comprises anthocyanins and the anthocyanins are present in the composition at a concentration of at least 25 weight-%, preferably at least 30 weight-%, or at least 35 weight-%, or at least 40 weight-%, or at least 45 weight-%, or at least 50 weight-%. It is preferred, according to the present invention, when the extract is an alcoholic extract, preferably a methanol extract.

The invention also covers a method of making the medical device as described, the method comprising applying the coating composition to the exterior surface of the medical device, optionally wherein the coating composition is formulated as a cream, a hydrogel cream, or a spray.

Moreover, the invention refers to a deep-lung particle comprising a composition comprising an extract of black currants and bilberries, which is dispensed into the deeper respiratory tract of an individual and a device for dispensing a deep-lung particle into the deeper respiratory tract of an individual.

The composition may comprise a formulation of extracts of black currants and bilberries with nanoparticles, preferably liposomes. Such formulations may be inhaled to maximize the delivery of nanoparticles into the lung. Inhalation facilitates the localized delivery of compositions directly to the lungs via the oral or nasal inhalation route. For example, aerosolized delivery of liposomal interleukin-2 (IL-2) in dogs has been shown to be effective against pulmonary metastases from osteosarcoma (Khanna C, Anderson P M, Hasz D E, Katsanis E, Neville M, Klausner J S. Interleukin-2 liposome inhalation therapy is safe and effective for dogs with spontaneous pulmonary metastases. Cancer 1997; 79: 1409-21.) Moreover, the delivery of anticancer drugs via nanoparticles has been shown to be efficacious and safe in a variety of cancers. Anticancer drugs can also be formulated into drug nanocrystals with high drug loading and minimal use of excipients. (Sharad M, Wei G, Tonglei L, Qi Z, Review: Pulmonary delivery of nanoparticle chemotherapy for the treatment of lung cancers: challenges and opportunities, Acta Pharmacologica Sinica (2017) 38: 782-797).

In a preferred embodiment, a nanoparticle suspension comprising the composition according to the present invention is aerosolized into droplets with appropriate aerodynamic diameters using currently available inhalation devices. Such inhalation devices are preferably selected from nebulizers and pressurized metered dose inhalers (pMDI).

Therefore, in an advantageous configuration, the composition according to the present invention may also be formulated as nanoparticle suspension for use in a nebulizer. Such nebulizers convert suspension of nanoparticles into inhalable droplets and may be used for the delivery of the composition into the deep lungs without compromising liposome integrity. An alternative configuration refers to pMDIs, which create small inhalable droplets of drugs suspended in compressed propellant (such as hydrofluoroalkane (HFA)).

The present invention also refers to a nanoparticle formulation as a dry powder, which offers greater long-term stability than a suspension. Controlling the size of nanoparticles is central for their formulation into reliable and efficient inhalable dry powders. Nanoparticles can be dried with/without excipients via spray-drying, freeze-drying and spray freeze-drying to generate stable and uniformly sized inhalable particles.

In an alternative embodiment, nanoparticles may be co-dried with excipients, which leads to the formation of inhalable nanoparticle aggregates in an excipient matrix. It is possible to utilize particle engineering and ensure consistent and highly efficient delivery of nanoparticles to the lungs through nano-aggregates, large porous particles, and other formulation techniques.

The activity of the composition comprising an extract of black currants and bilberries described herein against viruses from the Herpesviridae family may also be utilized in the context of cell culture and cell storage ex vivo, and in particular in the preparation of cells for cell therapy. Accordingly, the present invention also provides a method for preventing or reducing the risk of a virus infection in a cell or cells ex vivo comprising contacting the cell or cells with a composition comprising an extract of black currants and bilberries, optionally wherein the cell or cells are stem cells or CAR T cells, optionally wherein the contacting comprises culturing or storing the cell or cells with the composition. In particular, the composition may be added directly to the cells or added to cell media or to another composition which is then added to the cells. The extract or black currants and bilberries may be as described above for the other aspects of the invention.

Item List

Preferred embodiments of the present invention are summarized in the following item list:
1. A composition for use in treating or preventing a virus infection in a subject, wherein the virus is from the Herpesviridae family and wherein the composition comprises an extract of black currants and bilberries.
2. The composition for use according to item 1, wherein the black currants are the fruit of Ribes nigrum and/or the bilberries are the fruit of Vaccinium myrtillus.
3. The composition for use according to any preceding item wherein the composition contains an extract from black currants and bilberries in a weight ratio of 0.5:1 to 1:0.5.
4. The composition for use according to any preceding item wherein the composition is an extract of the pomaces from black currants and bilberries.
5. The composition for use according to any preceding item, wherein the composition comprises anthocyanins and the anthocyanins are present in the composition at a concentration of at least 25 weight-%.
6. The composition for use according to any preceding item, wherein the extract is an alcoholic extract, preferably a methanol extract.
7. The composition for use according to any preceding item, wherein the extract is prepared by a process comprising the steps of extraction of black currants and/or bilberries, purification via chromatography, mixing of the extract(s) with water and spray-drying of the mixture.
8. The composition for use according to any preceding item wherein the composition comprises one or more of the following anthocyanins: cyanidin-3-glucoside, cyanidin-3-galactoside, cyanidin-3-arabinoside, delphinidin-3-glucoside, delphinidin-3-galactoside, delphinidin -3-arabinoside, petunidin-3-glucoside, petunidin-3-galactoside, petunidin-3-arabinose, peonidin-3-glucoside, peonidin-3-galactoside, peonidin-3-arabinose, malvidin-3-glucoside, malvidin-3-galactoside, malvidin-3-arabinose, cyanidin-3-rutinoside, delphinidin-3-rutinoside.
9. A composition for use in treating or preventing a virus infection in a subject, wherein the virus is from the Herpesviridae family and wherein the composition comprises: cyanidin-3-glucoside, cyanidin-3-rutinoside, delphinidin-3-glucoside, delphinidin-3-rutinoside, cyanidin-3-galactoside and delphinidin-3-galactoside.
10. The composition for use according to any preceding item wherein the virus is from the sub-family Alphaherpesvirinae or Gammaherpesvirinae.
11. The composition for use according to any preceding item wherein the virus is herpes simplex virus-1 (HSV-1), herpes simplex virus-2 (HSV-2), Varicella zoster virus (VZV), Epstein—Barr virus (EBV), Cytomegalovirus (CMV), Roseolovirus, or Kaposi's sarcoma-associated herpesvirus (KSHV, HHV-8).
12. The composition for use according to the previous item wherein the virus is HSV-1, EBV, CMV or HHV-8 preferably wherein the virus is HSV-1, CMV or HHV-8.
13. The composition for use according to any preceding item wherein the composition suppresses viral infection.
14. The composition for use according to any preceding item wherein the composition comprises anthocyanins and is to be administered to the subject 1 to 10 oral dosages of at least 80 mg anthocyanins each per day, preferably 3 to 6 oral dosages of at least 80 mg anthocyanins each per day.
15. The composition for use according to any preceding item wherein the composition is to be administered to the subject as parenteral bolus injection or infusion or parenteral nutritional solution to stabilize critical patients.
16. The composition for use according to any preceding item wherein the composition is to be administered to the subject, reaching a concentration in the target compartment of at least 30 μg/ml, preferably at least 100 μg/ml.
17. The composition for use according to any preceding item, wherein the composition is for use with a medical device which is to be inserted into the subject, or wherein the subject has had a medical device inserted, optionally wherein the inserted device is transdermal or endotracheal.
18. The composition for use according to item 17, wherein the composition is to be administered at a site of insertion of the medical device into the subject.
19. The composition for use according to item 17 or 18, wherein the medical device is for endotracheal intubation or parenteral nutrition.

20. The composition for use according to any of item 17 to 19, wherein the medical device is a needle, a catheter, a port, an intubation device or tube, a nebulizer, an implant, a vascular access catheter, a brain microcatheter, a peripherally inserted central catheter, a chronic central venous catheter, an implanted port, an acute central venous catheter, a midline catheter, a short peripheral intravenous catheter, or a dialysis catheter.

21. The composition for use according to any of item 17 to 20, wherein a dwell time of the medical device in the subject is more than 24 hours, more than 48 hours, more than 72 hours, more than one week, more than 2 weeks, more than 3 weeks, preferably wherein the dwell time is more than one week, more than 2 weeks or more than 3 weeks.
22. The composition for use according to any preceding item wherein the subject is a human.
23. The composition for use according to any preceding item wherein the subject is pregnant.
24. The composition for use according to any preceding item wherein the subject is a carrier of a virus from the Herpesviridae family, preferably wherein the subject is a carrier of herpes simplex virus.
25. The composition for use according to any preceding item wherein the subject is infected with Kaposi's sarcoma-associated herpesvirus (KSHV, HHV-8), optionally wherein the subject is HIV-positive or is suffering from AIDS.
26. The composition for use according to any preceding item wherein the virus infection is in the liver or kidney.
27. The composition for use according to any preceding item for the prevention or treatment of a cancer associated with a virus from the Herpesviridae family, optionally wherein:
   (i) the virus is EBV and the cancer is lymphoma (including Hodgkin lymphoma and Burkitts lymphoma), nasopharyngeal cancer, gastric cancer, or breast cancer; or
   (ii) the virus is HHV-8 and the cancer is Kaposi's sarcoma, primary effusion lymphoma, HHV-8-associated multicentric Castleman disease, or breast cancer.
28. The composition for use according to any preceding item for the prevention or treatment of an autoimmune disease associated with a virus from the Herpesviridae family, optionally wherein:
   (i) the virus is EBV and the autoimmune disease is systemic lupus erythematosus (SLE), rheumatoid arthritis (RA), Sjogren's syndrome or multiple sclerosis; or the virus is HSV-1 and the autoimmune disease is multiple sclerosis.
29. The composition for use according to any preceding item, wherein the subject is immunocompromised.
30. The composition for use according to the previous item, wherein the subject is taking an immunosuppressant.
31. The composition for use according to any preceding item, wherein the subject is exposed to physical or emotional stress.
32. The composition for use according to the preceding item, wherein the subject is suffering from fatigue, depression or anxiety.
33. The composition for use according to the preceding item for the prevention or treatment of Alzheimer disease.
34. The composition for use according to claim 33, wherein the composition reduces β-amyloid plaque formation, optionally wherein the composition reduces β-amyloid plaque formation by reducing or preventing a virus infection.
35. The composition for use according to claim 33 or claim 34, wherein the composition reduces brain tissue inflammation.
36. A topical composition comprising an extract of black currants and bilberries, wherein the composition further comprises a pharmaceutically acceptable excipient suitable for a topical composition that is to be administered to the skin, preferably wherein the pharmaceutically acceptable excipient comprises one or more of a tonicity adjusting agent, a buffering agent, a preservative, an antioxidant, a stabilizer, a pH adjusting agent, a penetration enhancer, a surfactant and a humectant.
37. An eye drop composition comprising an extract of black currants and bilberries, wherein the composition further comprises a pharmaceutically acceptable excipient suitable for a composition that is to be administered to the eye, preferably wherein the pharmaceutically acceptable excipient comprises one or more of a tonicity adjusting agent, a buffering agent, a preservative, an antioxidant, a stabilizer, a pH adjusting agent, a penetration enhancer, a surfactant and a humectant.
38. A composition comprising an analgesic or anti-inflammatory agent and an extract of black currants and bilberries, preferably wherein the analgesic is ibuprofen or paracetamol/acetaminophen.
39. A composition according to the previous item for use in treating pain associated with a virus infection in a subject, wherein the virus is from the Herpesviridae family.
40. A combined preparation comprising an analgesic, and an extract of black currants and bilberries, for simultaneous, separate or sequential use in medicine.
41. A topical composition comprising an analgesic, and an extract of black currants and bilberries.
42. The composition according to any of items 36 to 41, wherein the composition comprises anthocyanins and the anthocyanins are present in the composition at a concentration of at least 25 weight-%.
43. A medical device suitable for insertion into a subject, the medical device comprising a coating composition on an exterior surface of the device, wherein the coating composition comprising an extract of black currants and bilberries.
44. The medical device according to item 43, wherein the medical device is a needle, a catheter, a port, an intubation device or tube, a nebulizer, an implant, a vascular access catheter, a brain microcatheter, a peripherally inserted central catheter, a chronic central venous catheter, an implanted port, an acute central venous catheter, a midline catheter, a short peripheral intravenous catheter, or a dialysis catheter, preferably wherein the exterior surface of the medical device is plastic.
45. A method of making the medical device according to item 43 or 44, the method comprising applying the coating composition to the exterior surface of the medical device, optionally wherein the coating composition is formulated as a cream, a hydrogel cream, or a spray.

46. A composition comprising an antiviral agent, and an extract of black currants and bilberries, wherein the antiviral agent is a Herpesviridae antiviral agent, preferably wherein the antiviral agent is an inhibitor of DNA replication, optionally wherein the antiviral agent is a DNA polymerase inhibitor or a DNA terminase complex inhibitor.
47. The composition of item 46, wherein the antiviral agent is acyclovir, ganciclovir, valganciclovir, foscarnet, famciclovir, penciclovir, valaciclovir, or letermovir.
48. The composition according to item 46 or 47 which is in the form of a topical composition or eye drops, preferably wherein the antiviral agent is acyclovir.

49. A combined preparation comprising an antiviral agent, and an extract of black currants and bilberries, for simultaneous, separate or sequential use in medicine.
50. A method for preventing or reducing the risk of a virus infection in a cell or cells ex vivo comprising contacting the cell or cells with a composition comprising an extract of black currants and bilberries, optionally wherein the cell or cells are stem cells or CAR T cells, optionally wherein the contacting comprises culturing or storing the cell or cells with the composition.
51. A method for treating or preventing a virus infection in a subject in need thereof comprising administering to the subject an effective amount of a composition comprising an extract of black currants and bilberries, wherein the virus is from the Herpesviridae family.
52. A method for suppressing a virus infection or preventing virus reactivation in a subject in need thereof, comprising administering to the subject an effective amount of a composition comprising an extract of black currants and bilberries, wherein the virus is from the Herpesviridae family.
53. A method for preventing a device-associated virus infection in a subject, comprising: (a) inserting a device into the subject and administering an effective amount of a composition comprising an extract of black currants and bilberries at a site of insertion of the device; and/or (b) applying an effective amount of a composition comprising an extract of blackcurrants and bilberries to an external surface of a device and inserting the device into the subject,

wherein the virus is from the Herpesviridae family.
54. A method for treating or preventing a cancer associated with a virus from the Herpesviridae family in a subject in need thereof, comprising administering to the subject an effective amount of a composition comprising an extract of black currants and bilberries.
55. A method for treating or preventing an autoimmune disease associated with a virus from the Herpesviridae family in a subject in need thereof, comprising administering to the subject an effective amount of a composition comprising an extract of blackcurrants and bilberries.
56. A method for reducing β-amyloid plaque formation and/or reducing brain tissue inflammation in a subject in need thereof, comprising administering to the subject an effective amount of a composition comprising an extract of blackcurrants and bilberries, optionally wherein the composition reduces β-amyloid plaque formation and/or brain tissue inflammation by reducing or preventing an infection by a virus from the Herpesviridae family.
57. The method according to any of items 51 to 56 wherein the extract is as defined in any of items 2 to 7.
58. The method according to any of items 51 to 57, wherein the virus is as defined in item 8.
59. The method according to any of items 51 to 58, wherein the composition is to be administered as defined in items 9 or 10.
60. The method according to any one of items 51 to 59, wherein the subject is as defined in any of items 16 to 18.

### EXAMPLES

The berry extracts composition (Healthberry® 865; Evonik Nutrition & Care GmbH, Darmstadt, Germany) used in the present study is a dietary supplement consisting of 17 purified anthocyanins (all glycosides of cyanidin, peonidin, delphinidin, petunidin, and malvidin) isolated from black currant (Ribes nigrum) and bilberries (Vaccinium myrtillus).

The relative content of each anthocyanin in the Healthberry® 865 product was as follows: 33.0% of 3-O-b-rutinoside, 3-O-b-glucosides, 3-O-b-galactosides, and 3-O-b-arabinosides of cyanidin; 58.0% of 3-O-b-rutinoside, 3-O-b-glucosides, 3-O-b-galactosides, and 3-O-b-arabinosides of delphinidin; 2.5% of 3-O-b-glucosides, 3-O-b-galactosides, and 3-O-b-arabinosides of petunidin; 2.5% of 3-O-b-glucosides, 3-O-b-galactosides, and 3-O-arabinosides of peonidin; 3.0% of 3-O-b-glucosides, 3-O-b-galactosides, and 3-O-b-arabinosides of malvidin.

The 3-O-b-glucosides of cyanidin and delphinidin constituted at least 40-50% of the total anthocyanins.

The major anthocyanins contained in the berry extract used are cyanidin-3-glucoside, cyanidin-3-rutinoside, delphinidin-3-glucoside, delphinidin-3-rutinoside, cyanidin-3-galactoside and delphinidin-3-galactoside.

In addition to the anthocyanins mentioned above, the product also contained maltodextrin (around 40 weight-% of the composition), and citric acid (to maintain stability of anthocyanins). The amount of anthocyanin citrate is at least 25 weight-% of the composition. The composition is prepared from black currants and bilberries by a process comprising the steps of alcoholic extraction of black currants and bilberries, purification via chromatography, mixing of the extracts with maltodextrin citrate and water and spray-drying of the mixture. The product composition contains extracts of black currants and bilberries mixed in a weight ratio of around 1:1.

Materials:

TABLE 1
Materials used for the measurement of cell survival and metabolism

| Material | Supplier |
|---|---|
| RealTime-Glo ™ MT Cell Viability | Promega GmbH, Mannheim (Germany) |
| Assay | |
| CellTiter-Glo ® Luminescent Cell | Promega GmbH, Mannheim (Germany) |
| Viability Assay | |
| Dulbecco′s Modified Eagle′s medium | Gibco Life technologies, Carlsbad (USA) |
| (DMEM) | |
| Fetal bovine serum | Gibco Life technologies, Carlsbad (USA) |
| BHK cells | ATCC/American Type Culture Collection in |
| | Partnership with LGC standards, Wesel (Germany) |
| Healthberry ® 865(anthocyanin | Evonik Nutrition & Care GmbH, Darmstadt |
| content 29.7%) | (Germany) |

TABLE 2
Devices used for the measurement of cell survival and metabolism.

| Device | Supplier |
|---|---|
| Centro LB 960 microplate | Berthold Technologies, (Germany) |
| luminometer | |

TABLE 3
Materials used for anti-viral assay

| Material | Supplier |
|---|---|
| Wildtype HSV-1 virus, herpes simplex | Institute of Virology, Würzburg (Germany) |
| virus 1 | |
| Influenza virus serotype A | patient derived isolate, Institute of Virology |
| | Würzburg (Germany) |
| HHV-8 | Brune, HPI, Hamburg (Germany) |
| mCMV | Institute of Virology, Würzburg (Germany) |
| Dulbecco′s Modified Eagle′s medium | Gibco Life technologies, Carlsbad (USA) |
| (DMEM) | |
| Fetal bovine serum | Gibco Life technologies, Carlsbad (USA) |
| BHK cells | ATCC/American Type Culture Collection in |
| | Partnership with LGC standards, Wesel (Germany) |
| MDCK cells | ATCC/American Type Culture Collection in |
| | Partnership with LGC standards, Wesel (Germany) |
| NIH3T3 | ATCC/American Type Culture Collection in |
| | Partnership with LGC standards, Wesel (Germany) |
| HP Viral Nucleic Acid Kit | Hoffman-La-Roche Ltd., Basel (Switzerland) |
| RTqPCR LightMix ® Modular Influenza | Hoffman-La-Roche Ltd., Basel (Switzerland) |
| A kit (Cat. No. 07 792 182 001) | |
| LightCycler ® Multiplex RNA Virus | Hoffman-La-Roche Ltd., Basel (Switzerland) |
| Master kit (Cat. No. 07 083 173 001) | |
| Healthberry ® 865 (anthocyanin | Evonik Nutrition & Care GmbH, Darmstadt |
| content 29.7%) | (Germany) |
| Bilberry extract, Vaccinium myrtillus | Evonik Nutrition & Care GmbH, Darmstadt |
| (anthocyanin content 38.8%) | (Germany) |
| Black currant extract, Ribes nigrum | Evonik Nutrition & Care GmbH, Darmstadt |
| (anthocyanin content 30%) | (Germany) |
| Berry extract analogue to | Evonik Nutrition & Care GmbH, Darmstadt |
| Healthberry ® 865 without maltodextrin | (Germany) |
| GLUCIDEX IT 19 (maltodextrin) | ROQUETTE GmbH, Frankfurt (Germany) |
| Delphinidin 3-rutinoside/D3R | Polyphenols AS, Sandnes (Norway) |
| Delphinidin 3-glucoside/D3G | Polyphenols AS, Sandnes (Norway) |
| Cyanidin 3-rutinoside/C3R | Polyphenols AS, Sandnes (Norway) |
| Cyanidin 3-glucoside/C3G | Polyphenols AS, Sandnes (Norway) |
| Delphinidin 3-galactoside/D3Gal | Polyphenols AS, Sandnes (Norway) |
| Petunidin 3-glucoside/Pet3G | Polyphenols AS, Sandnes (Norway) |

TABLE 4
Materials used for β-Amyloid assay

| Material | Supplier |
|---|---|
| Wildtype HSV-1 virus, herpes simplex | Institute of Virology, Würzburg (Germany) |
| virus 1 | |
| Healthberry ® 865 (anthocyanin | Evonik Nutrition & Care GmbH, Darmstadt |
| content 29.7%) | (Germany) |
| Berry extract analogue to | Evonik Nutrition & Care GmbH, Darmstadt |
| Healthberry ® 865 without maltodextrin | (Germany) |
| GLUCIDEXIT 19 (maltodextrin) | ROQUETTE GmbH, Frankfurt (Germany) |
| SH-SY5Y (neuroblastoma) cells | ATCC/American Type Culture Collection in |
| | Partnership with LGC standards, Wesel (Germany) |
| rabbit polyclonal anti-beta amyloid 1- | Abcam,Cambridge (UK) |
| 42, β-Amyloid, #ab10148 | |
| polyclonal goat anti-green fluorescent | Rockland Immunochemicals, Pottstown (USA) |
| protein, GFP, #600-101-215 | |
| donkey anti-rabbit AlexaFluoR ® 555 | Thermo Fisher Scientific GmbH, Dreieich |
| secondary antibody, #A-31572 | (Germany) |
| donkey anti-goat AlexaFluor ® 647 | Thermo Fisher Scientific GmbH, Dreieich |
| secondary antibody, #A-21447 | (Germany) |
| Fluoromount-G ™ TM with DAPI, | Thermo Fisher Scientific GmbH, Dreieich |
| #00-4959-52 | (Germany) |
| Dulbecco′s Modified Eagle′s medium | Gibco Life technologies, Carlsbad (USA) |
| (DMEM) | |
| Fetal bovine serum | Gibco Life technologies, Carlsbad (USA) |

TABLE 5
Devices used for the anti-viral assay

| Device | Supplier |
|---|---|
| LightCycler96 qPCR 20 | Hoffman-La-Roche Ltd., |
| machine | Basel (Switzerland) |
| Lighcylcler96 Application | Hoffman-La-Roche Ltd., |
| software V1.1 | Basel (Switzerland) |
| PerkinElmer Ensight system | Perkin Elmer, Rodgau (Germany) |

Methods:

Test Compound Preparation:

All test compounds were dissolved and diluted in cell culture medium. The overall amount of anthocyanins was normalized between Healthberry® 865 and the single anthocyanins (e.g. 500 μg/mL of Healthberry® 865 corresponds to 150 μg/mL of anthocyanins tested for the single test compounds) or as well the single berry extracts (taken into account that Healthberry® 865 also contains maltodextrin besides the anthocyanins). The medium served as control for viral inhibition or cytotoxicity.

Cell Viability Assay:

Cell viability was measured by RealTime-Glo™ MT Cell Viability Assay (Cat. No. G9712, Promega, Germany). BHK cells were incubated with decreasing amounts of the compound solubilized in DMEM. Wells with DMEM alone served as control. The MT Cell Viability Substrate and the NanoLuc® luciferase were added according to the manufacturer's instructions. The assays were performed in triplicates. After 3 days the luminescence signal was measured with Centro LB 960 microplate luminometer (Berthold Technologies, Germany). Luminescence values after 1 h were set to 1 and changes over time were determined.

Anti-Viral Assay:

Herpes virus infection:

BHK cells were incubated with decreasing concentration of the solubilized test compounds for approx. 1 h. All concentrations were analyzed by six independent replicates on a black 96-well plate (PerkinElmer). Cells were infected with GFP-encoding wildtype HSV-1 virus and incubated for two days. Two days after infection, HSV-1-infected cells and GFP expressing cells were directly counted using the PerkinElmer Ensight system with optical cell culture plates. The instrument was controlled by manual counting. Anti-viral assays for HHV8 and mCMV were performed accordingly.

To not only analyze the virus entry and early phase of virus replication of infection but also later phases of viral replication, the test assay was adjusted accordingly. BHK cells were incubated with test compounds and subsequently infected with HSV-1. Two days after infection supernatants were collected, centrifuged to remove detached cells and used to infect BHK cells. After two additional days infected cells were quantified using the Ensight system.

From the first identification till now, antiviral compounds are initially identified via screening assay either in vitro or in cell culture using replication assays. Even the activities of compounds identified by in vitro enzyme screening tests need to be verified in cell culture-based assays. These assays are state of the art methods to identify and confirm antiviral activities since they allow the quantification of the inhibition of viral replication and ensure the cellular uptake of compounds. For example, aciclovir, the gold standard in the treatment of HSV-1, was identified by screening of antiviral substances in sponges (Elion et al., 1977 Selectivity of action of an antiherpetic agent, 9-(2-hydroxyethoxymethyl)guanine. PNAS 74. 5716). Later, the antiviral activity of aciclovir inhibiting other members of the Herpesviridae was shown in cell culture-based assays as well (AKESSON-JOHANSSON et al., 1990 Inhibition of Human Herpesvirus 6 Replicationby9-[4-Hydroxy-2-(Hydroxymethyl)Butyl]Guanine (2HM-HBG) and Other Antiviral Compounds. AAC 34. 2417). Moreover, all compounds used as clinical drugs against HIV-1, such as 3TC and Lopinavir (ABT-378), were initially tested in vitro to demonstrate their antiviral effects (Coates et al., 1992. The Separated Enantiomers of 2′-Deoxy-3′-Thiacytidine (BCH 189) Both Inhibit Human Immunodeficiency Virus Replication In Vitro. AAC 36. 202; Sham et al. 1998. ABT-378, a Highly Potent Inhibitor of the Human Immunodeficiency Virus Protease. AAC 42. 3218).

Influenza Genome Determination:

MDCK cells were seeded in 48 well plates. After 24 h test compounds were added, and cells were subsequently infected with influenza A virus. All infections were performed in triplicates. Cell culture supernatants were harvested three days post-infection and centrifuged at 2000 rpm to remove detached cells and analyze viruses secreted to the supernatant. Viral RNAs were isolated from 200μl cell culture supernatants using the Roche HP Viral Nucleic Acid Kit according to the manufacturer's manual. Viral genome copy numbers were determined using 5 μl of the eluted RNA and the RTqPCR LightMix® Modular Influenza A kit (Cat. No. 07 792 182 001, Roche) in combination with the LightCycler® Multiplex RNA Virus Master kit (Cat. No. 07 083 173 001, Roche). All PCR reactions were performed in triplicates from a RNAs with a Roche LightCycler96 qPCR 20. The Cq values were determined with the respective cycler software (Roche Lighcylcler96 Application software V1.1). The internal standard of the Modular Influenza A kit with 1000 genome copies served as positive control. Quality was ensured by following the MIQE guidelines.

β-Amyloid Assay: Analysis of Intracellular Deposition of β-Amyloid

SH-SY5Y (neuroblastoma) cells (5,000 per well) were incubated with the following samples including HHV-1 (MOI=8-10): Healthberry® 865 alone (500, 250 and 125 μg/ml) and Healthberry® 865 (500, 250 and 125 μg/ml) in combination with HHV-1, whereas HHV-1 alone and maltodextrin alone served as positive and negative controls.

Samples were fixed using Histofix and permeabilized with 0.05% Triton X-100 in phosphate-buffered saline for 5 min. Blocking steps were performed with 3% bovine serum albumin for 1 h followed by primary antibody incubation with rabbit polyclonal anti-beta amyloid 1-42 and polyclonal goat anti-green fluorescent protein. Antibodies were diluted 1:100 and 1:1000, respectively, and were incubated overnight. To visualize β-Amyloid antibodies, donkey anti-rabbit AlexaFluor® 555 secondary antibodies were used (diluted 1:400); to detect the GFP antibodies donkey anti-goat AlexaFluor® 647 secondary antibodies (diluted 1:400) were used. The samples were then mounted in Fluoromount-G™ with DAPI and analyzed with the BZ-9000 BIOREVO System (Keyence).

Anti-Viral Human Case for Epstein-Barr Virus

For the human case one individual displayed symptoms of fever, sore throat and swollen lymph nodes. After three days of symptoms Ibuprofen treatment was started with 2-3 times tablets per day, each with 400 mg dose. Furthermore, treatment with two times 80 mg of Healthberry® 865 (80 mg dose corresponds to the dose of one Medox® capsule) was started with administration in the morning as well as in the evening; Healthberry® 865 powder was solved in water to facilitate oral administration. After additional three days blood parameters as well as EBV antibody levels were analysed. This analysis of blood parameters was repeated after 9 days as well as 25 days. Within the first week treatment with Ibuprofen in combination with Healthberry® 865 was continued and for the following ˜2 weeks treatment was reduced to Healthberry® 865.

### Example 1

### Influence of Berry Extracts on Cell Viability

To exclude cellular toxicity and adverse side effects, cellular viabilities of the test compounds on BHK cells (96-well-plate: 650 cells/well) were determined with the RealTime-Glo™ MT Cell Viability Assay kit. This assay measures the intracellular ATP content and therefore provides information on the cellular viability and metabolism. The cells were incubated with decreasing compound concentration in triplicate assays. Subsequently, both the MT Cell Viability Substrate and NanoLuc® Enzyme were added, and the luciferase activities were measured after 1 h. The luminescence was measured after three days and normalized on the mean of the medium control wells. These compensations result in values of 1 for the medium control and values less than 1 indicate a lower number of cells or a decrease in metabolic activity compared to the appropriate controls.

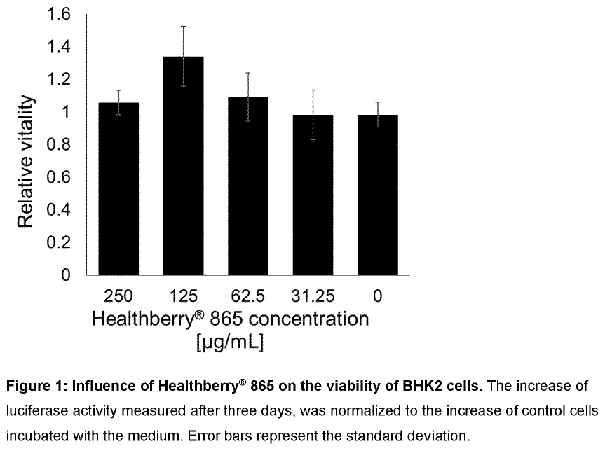

FIG. 1 displays the influence of Healthberry® 865 on the viability of BHK2 cells. The increase of luciferase activity measured after three days, was normalized to the increase of control cells incubated with the medium. Error bars represent the standard deviation.

Healthberry® 865 did not negatively influence cellular growth or metabolic activity at any concentration analysed, indicating the compound was non-toxic at these concentrations.

### Example 2

### Anti-viral effects of Healthberry® 865 on Herpes Simplex Virus 1

BHK cells were pre-incubated with decreasing concentrations of either Healthberry® 865 or with Healthberry® 865 without maltodextrin. The concentrations of material without maltodextrin were adjusted to 0.6 times of the sugar containing product to compensate for the 40% maltodextrin content of Healthberry® 865. Thus, comparable concentrations of anthocyanins were used. The cells were subsequently infected with GFP-encoding HSV at a multiplicity of infection of 2.5, and infected GFP-expressing cells were counted one day after infection using the PerkinElmer Ensight system. Both Healthberry® 865 and the berry extract analogue without maltodextrin suppressed viral infectivity about 2 log steps at Healthberry® 865 concentrations of >0.250 μg/mL. This inhibition of viral infectivity observed is in the range of common anti-viral pharmaceutical compounds and indicates that Herpes simplex is a prime target for berry extracts of black currants and bilberries, such as Healthberry® 865. The analysis of berry extract analogue without maltodextrin showed that a concentration of 150 μg/mL of the active substances (corresponding to 250 μg/mL Healthberry® 865) is sufficient for the suppression of HSV. Thus, the sugar is not required as potential co-factor for drug uptake.

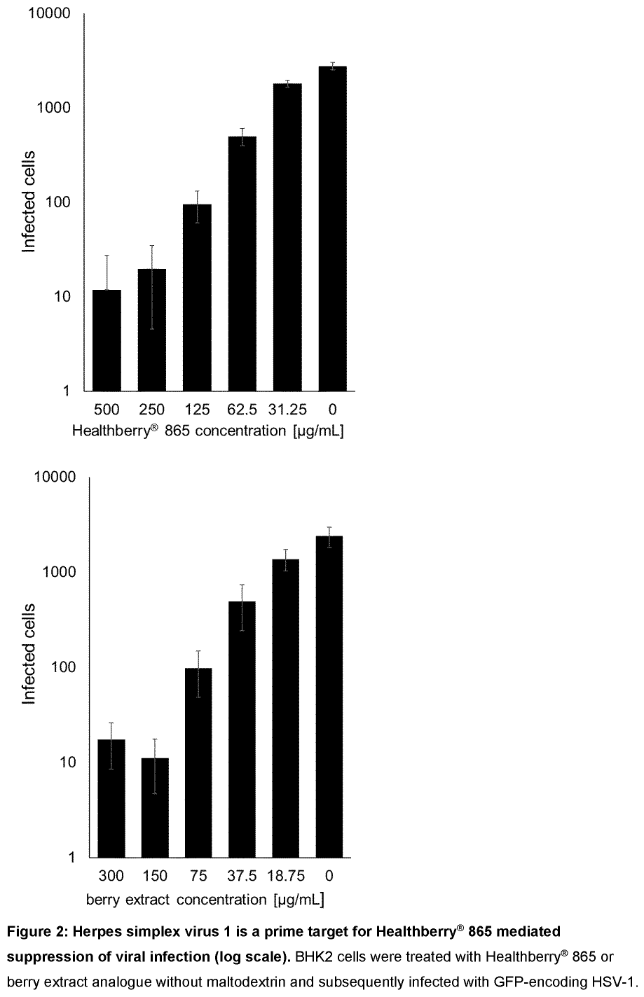

FIG. 2 shows that Herpes simplex virus 1 is a prime target for Healthberry® 865 mediated suppression of viral infection (log scale). BHK2 cells were treated with Healthberry® 865 or berry extract analogue without maltodextrin and subsequently infected with GFP-encoding HSV-1.

### Example 3

### Anti-viral effects of Healthberry® 865 on Influenza A Virus (Comparative)

The influence of Healthberry® 865 and single anthocyanins on the replication of Influenza A virus were analyzed. MDCK cells were incubated with the test compounds and subsequently infected with a patient-derived isolate of Influenza virus serotype A. All reactions were performed in triplicates. Cell culture supernatants were harvested after three days, and viral genomic RNAs were isolated from 200 μl cell culture supernatants. Viral loads were determined by RTqPCR using the LightMix® Modular Influenza A kit (Roche). Positive controls with 1000 Influenza genome copies were included in the RTqPCR. All RTqPCR reactions were performed in triplicates.

All test materials, including Healthberry® 865, showed similar amounts of virus in the supernatant as the negative control, with only minor differences indicating that none of the components inhibited influenza virus replication.

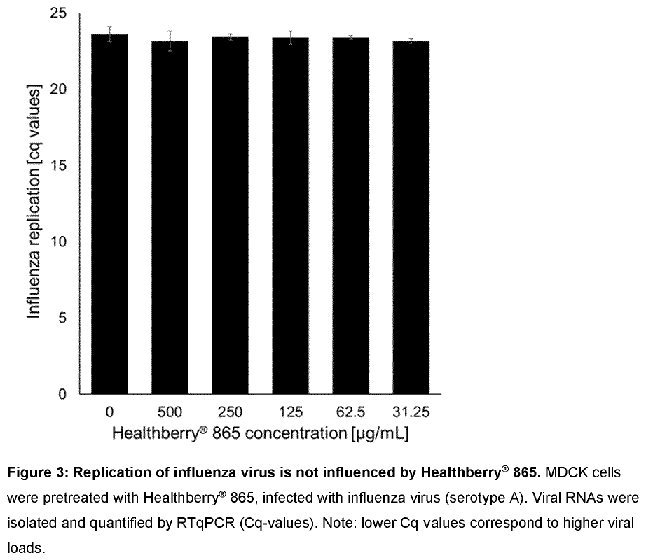

FIG. 3 shows that the replication of influenza virus is not influenced by Healthberry® 865. MDCK cells were pretreated with Healthberry® 865, infected with influenza virus (serotype A). Viral RNAs were isolated and quantified by RTqPCR (Cq-values; note: lower Cq values correspond to higher viral loads).

The results displayed no effect of Healthberry® 865 on Influenza A virus confirming the specificity of the anti-viral effects of berry extracts of black currants and bilberries on specific viruses or virus families, respectively. Other compounds as the single anthocyanins also did not show any influence on the replication of influenza virus.

### Example 4

### Anti-Viral Effects of Berry Extracts on Herpes Simplex Virus 1

Since Healthberry® 865 is a composition of bilberry and black currant extracts, it was analyzed, whether both extracts contain the compound active against HSV-1. BHK cells were incubated with 500, 250, and 125 mg/mL of Healthberry® 865, bilberry or black currant extract followed by infection with HSV-1. Two days after infection supernatants were collected, centrifuged to remove detached cells and used to infect BHK cells. After two additional days infected cells were quantified using the PerkinElmer Ensight system. The mean of infected cells from six independent wells was calculated. Error bars show the standard deviation.

Besides Healthberry® 865 both extracts showed viral inhibition indicating that the active compounds are present in both bilberry and black currant extracts. But in direct comparison with Healthberry® 865, bilberry and black currant extracts suppressed the HSV-1 viral infection to a lesser extent than Healthberry® 865, although especially the bilberry extract even contains about 10% more anthocyanins than Healthberry® 865. Especially in higher concentrations like 500 μg/mL bilberry and black currant extracts reached about 1.5 log scale reduction of viral infection whereas Healthberry® 865 surprisingly reached up to 2-3 log scales. The absolute values of infected cells emphasized the significance of the effect even more, with Healthberry® 865 reducing the number of infected cells from about 1 million to ˜300 (decrease to ˜0.3%), whereas the single extracts only reduce about 90000 infected cells down to 2200-3500 (decrease to ˜3%).

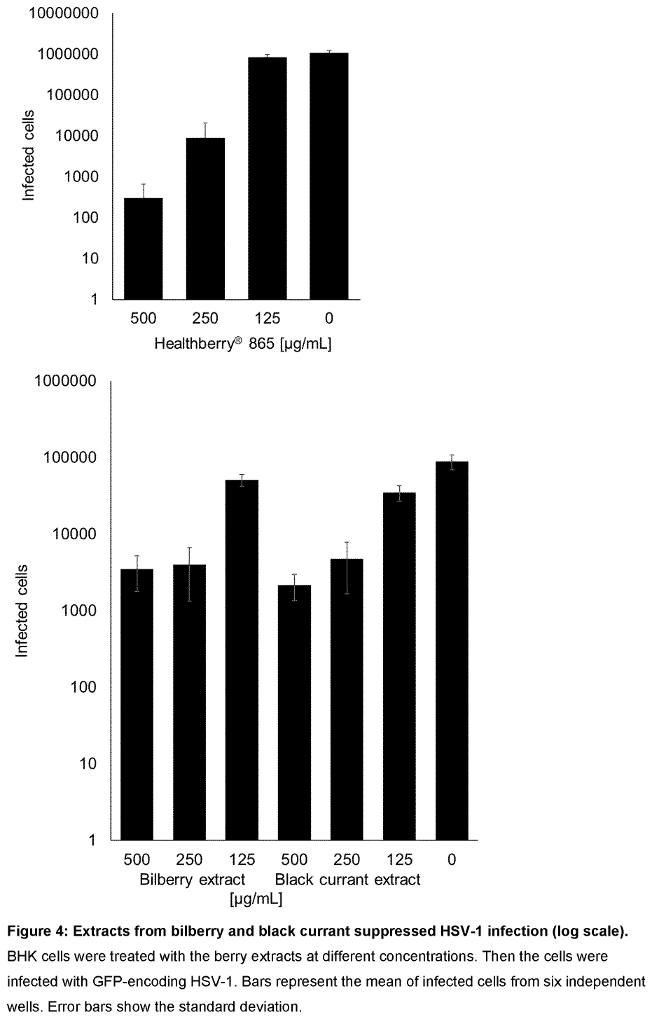

FIG. 4 shows that berry extracts from bilberry and black currant mediated suppression of viral infection (log scale). BHK cells were treated with black currant or bilberry extract and subsequently infected with GFP-encoding HSV-1.

### Example 5

### Anti-Viral Effects of Anthocyanins on Herpes Simplex Virus 1

To further identify the active compound of Healthberry® 865 several known anthocyanins were tested. Neither C3G nor D3Gal or Pet3G inhibited HSV-1, while D3G decreased viral infectivity like Healthberry® 865 providing evidence that D3G is an active HSV-1 inhibitor.

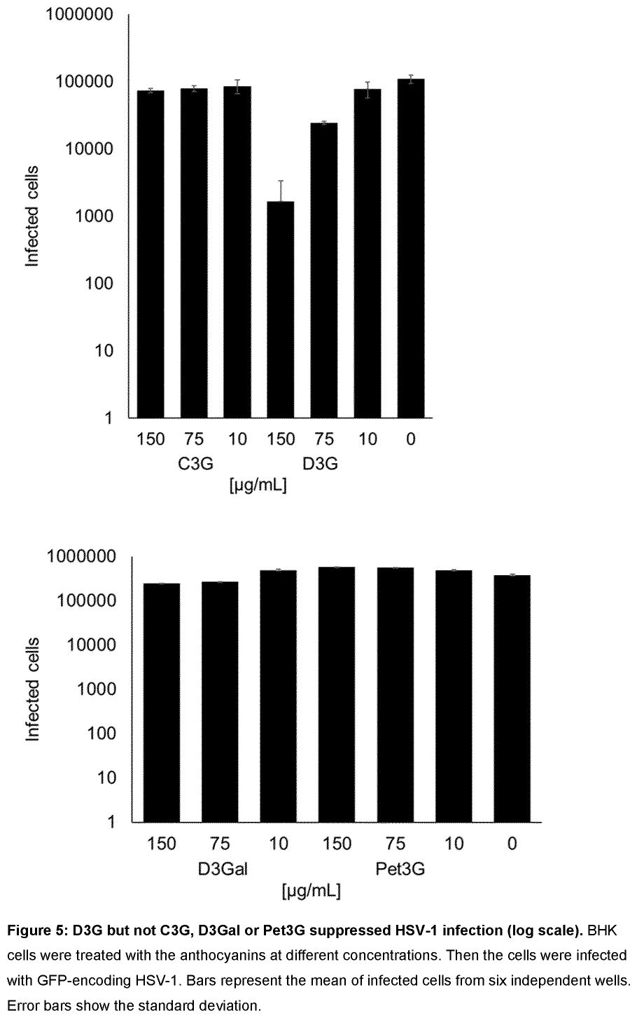

FIG. 5 shows that D3G, but not C3G, D3Gal or Pet3G, mediated suppression of viral infection (log scale). BHK cells were treated with anthocyanins and subsequently infected with GFP-encoding HSV-1.

### Example 6

### Reduction of HSV-1 Related Intracellular g-Amyloid Deposition by Healthberry® 865

Herpes virus infections and specifically HSV-1 infections are correlated to intracellular R-Amyloid and tau-protein deposition in cells of the CNS in progression of Alzheimer's disease. Thus, due to the significant anti-viral activity of Healthberry® 865 the influence of this berry extract on the protein deposition, and therefore plaques formation connected to Alzheimer's disease, was evaluated in an qualitative in vitro test system. Therefore, in a first step the infectability of SH-SYSY neuroblastoma cells with HHV-1 was tested. After incubation of SH-SYSY cultures with HHV-1 (MOI=8), an infection rate of >90% (qualitatively) was detected that came along with high intracellular β-Amyloid deposition. Negative controls (omission of secondary antibodies) showed only nuclear staining. These results confirmed SH-SYSY as suitable cell line for infection studies with HHV-1.

After treatment of SH-SYSY cells with Healthberry® 865 and subsequent infection with HHV-1 the number of both infected and β-Amyloid-positive cells significantly decreased with increasing Healthberry® 865 concentration (500, 250 and 125 μg/ml) qualitatively. For treatment of SH-SYSY cells with maltodextrin HHV-1-infected cells were mostly β-Amyloid-positive. These findings support previous data that maltodextrin does not contribute to the anti-viral activity and reduction of R-Amyloid by Health berry® 865.

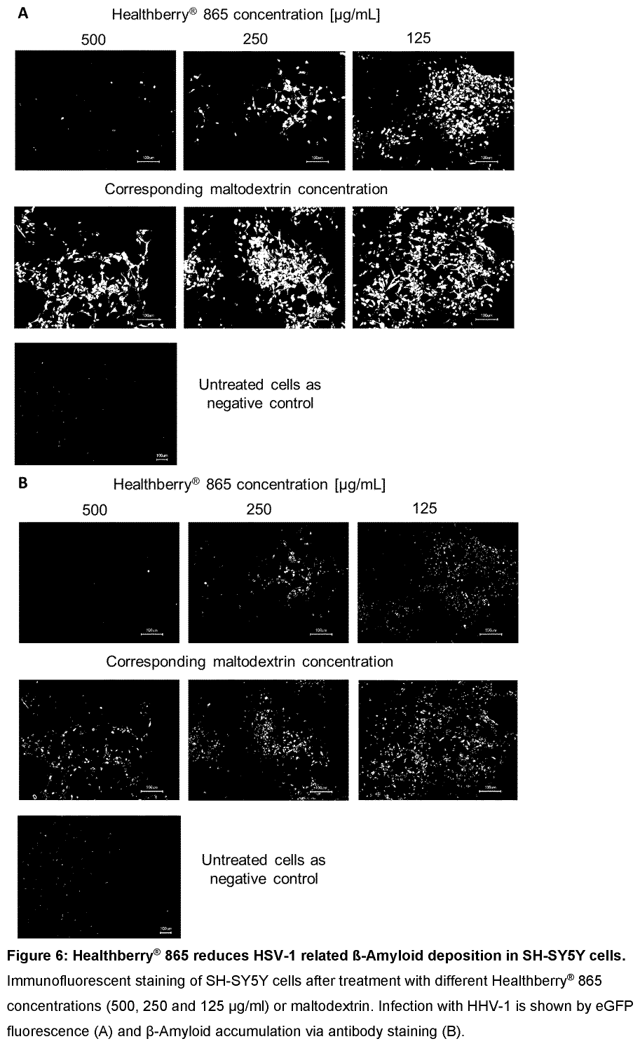

FIG. 6 shows that Healthberry® 865 reduces HSV-1 related R-Amyloid deposition in SH-SYSY cells Immunofluorescent staining of SH-SYSY cells was performed after treatment with different Healthberry® 865 concentrations (500, 250 and 125 pg/ml) or maltodextrin. Infection with HHV-1 and R-amyloid staining display a decrease in infected and β-Amyloid-positive cells when treated with Healthberry® 865.

### Example 7

### Anti-Viral Effects of Healthberry® 865, Berry Extracts & Anthocyanins on Herpes Virus 8/HHV8

Cells were pre-incubated with different concentrations of Healthberry® 865, berry extract analogue, bilberry extract, black currant extract or single anthocyanins. The concentrations of materials were again adjusted to the same levels of anthocyanins (except the single berry extracts). No treatment or only maltodextrin served as controls. The cells were subsequently infected with GFP-encoding HHV-8, and infected GFP-expressing cells were counted two days after infection using the PerkinElmer Ensight system.

Both Healthberry® 865 (two different lots) and the berry extract analogue without maltodextrin significantly suppressed viral infectivity up to two orders of magnitude. This inhibition of viral infectivity indicates that Herpes virus 8, and the family of Herpesviridae, is a target for

Healthberry® 865. The analysis of berry extract analogue without maltodextrin and the maltodextrin control confirmed again that the sugar moiety is not required as potential co-factor for drug uptake.

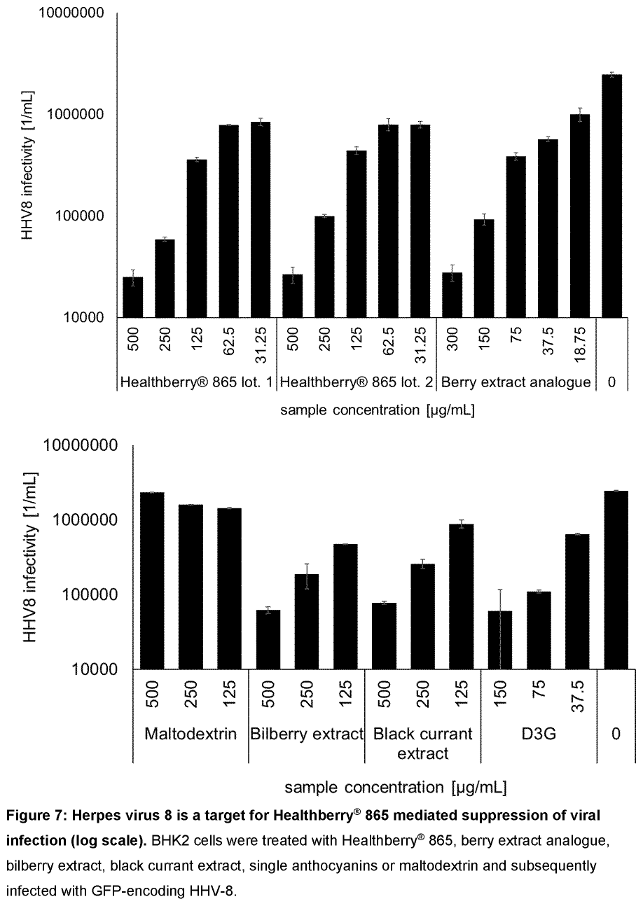

FIG. 7 shows that Herpes virus 8 is a target for Healthberry® 865 mediated suppression of viral infection (log scale). BHK2 cells were treated with Healthberry® 865, berry extract analogue without maltodextrin, bilberry extract, black currant extract, single anthocyanins or maltodextrin and subsequently infected with GFP-encoding HHV-8.

Besides Healthberry® 865 both single berry extracts, bilberry and black currant, showed viral inhibition as well as indication that the active compounds are present in both bilberry and black currant extracts. But in direct comparison with Healthberry® 865, bilberry and black currant extracts suppressed the HHV-8 viral infection again to a lesser extent than Healthberry® 865 (although especially the bilberry extract even contains about 10% more anthocyanins than Healthberry® 865), showing a synergistic effect of the extracts in the Healthberry® 865 mixture. The absolute values of infected cells again emphasized the significance of the effect, with Healthberry® 865 reducing the number of infected cells from about 2.5 million down to ˜25000 (decrease to 1%), whereas the single extracts only reduce the infected cells down to ˜60000-80000 (decrease to 2.8%). Furthermore, D3G could again be identified as an active ingredient in Healthberry® 865.

### Example 8

### Anti-Viral Effects of Healthberry® 865, Berry Extracts & Anthocyanins on Mouse Cytomegalovirus/mCMV

Cells were pre-incubated with different concentrations of Healthberry® 865, bilberry extract and black currant extract (no treatment served as control). The cells were subsequently infected with GFP-encoding mCMV, and infected GFP-expressing cells were counted two days after infection using the PerkinElmer Ensight system. Healthberry® 865 significantly suppressed viral infectivity up to 1.5 orders of magnitude. This inhibition of viral infectivity indicates that mouse cytomegalovirus, and the family of Herpesviridae, is a target for Healthberry® 865.

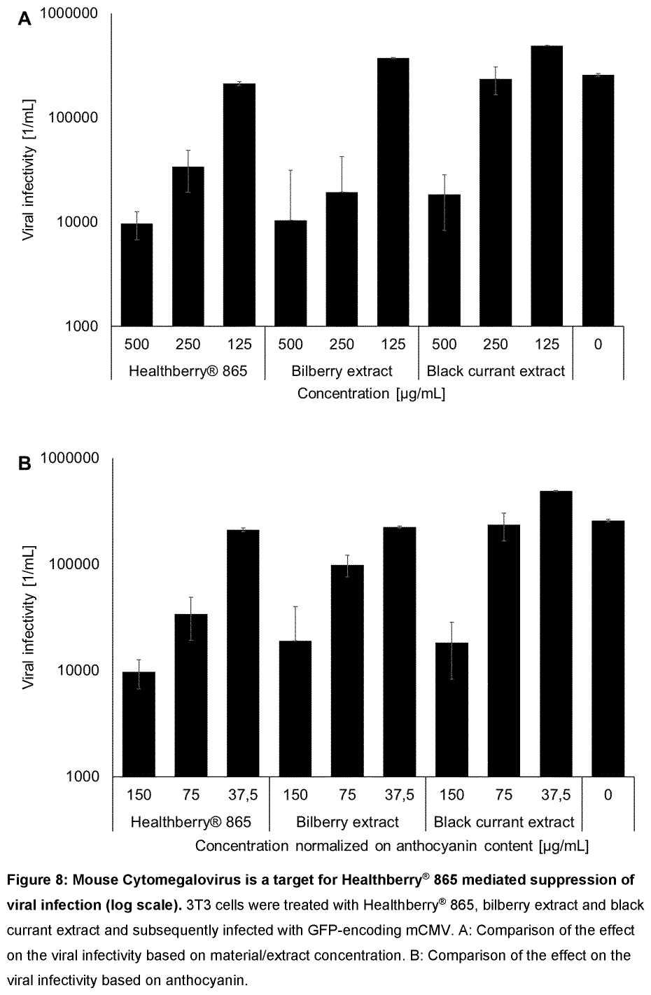

FIG. 8 shows that mouse Cytomegalovirus is a target for Healthberry® 865 mediated suppression of viral infection (log scale). 3T3 cells were treated with Healthberry® 865 or bilberry extract and black currant extract and subsequently infected with GFP-encoding mCMV.

Besides Healthberry® 865 both single berry extracts, bilberry and black currant, showed viral inhibition as well as indication that the active compounds are present in both bilberry and black currant extracts. In direct comparison with Healthberry® 865, bilberry and black currant extracts suppressed the mCMV viral infection again to a lesser extent, again indicating a synergistic effect of the extracts in the Healthberry® 865 mixture. As the synergistic effect is not that significant for mCMV as for hHSV-1 and hHHV-8, direct comparison of material concentrations in the assay did not show the difference in a proper way (FIG. 8A). In contrast, normalizing all the extracts, Healthberry® 865 as well as the single extracts, to their anthocyanin content (especially the bilberry extract contains about 10% more anthocyanins than Healthberry® 865), shows the synergistic effect of the extracts in the Healthberry® 865 mixture more obvious with a difference in the absolute values of the factor 2 (FIG. 8B). The absolute values of infected cells again emphasized the significance of the effect. In case of 150 μg/mL anthocyanin content Healthberry® 865 reduced the number of infected cells from about 250 thousand down to ˜9500, whereas the single extracts only reduce the infected cells down to ˜18500-19000; in case of 75 μg/mL Healthberry® 865 reduced the number of infected cells from about 250 thousand down to ˜34000, whereas the bilberry extract only reduced the infected cells down to ˜98500 and black currant extract even resulted in no reduction for this concentration.

These results confirm again the anti-viral effect of Healthberry® 865 on herpes viruses of the Herpesviridae family as the effect could even be transferred to mCMV, which is targeting mice as alternative species to humans.

### Example 9

### Human Case for Anti-Viral Effects of Healthberrv® 865 on Epstein-Barr Virus/EBV

For the human case one individual displayed symptoms of fever, sore throat and swollen lymph nodes. After three days of symptoms Ibuprofen treatment was started with 2-3 times tablets per day, each with 400 mg dose. Furthermore, treatment with two times 80 mg of Healthberry® 865 (80 mg dose corresponds to the dose of one Medox® capsule) was started with administration in the morning as well as in the evening; Healthberry® 865 powder was solved in water to facilitate oral administration. After additional three days blood parameters as well as EBV antibody levels were analysed. This analysis of blood parameters was repeated after 9 days as well as 25 days. Within the first week treatment with Ibuprofen in combination with Healthberry® 865 was continued and for the following ˜2 weeks treatment was reduced to Healthberry® 865.

After 6 days of symptoms (representing Day 0 in Table 6) the results of the blood analysis showed a significant increase of leukocytes, a decrease of thrombocytes and monocytes as well as an increase in all liver values. Furthermore, antibody levels for EBV displayed 30.5 U/mL EBV IgG 20 negative, >20 positive) and 78.9 U/mL for EBV IgM (<20 negative, 20-40 borderline, >40 positive) leading to the evident diagnosis of a fresh EBV infection.

TABLE 6
Blood parameters measured for EBV case individual.

| | | | | | | Significant |
|---|---|---|---|---|---|---|
| | expected | | Date of analysis | | | improvement |
| Blood | healthy | | Day | Day | Day | after |
| parameters | range | Unit | 0 | 9 | 25 | treatmernt |
| Leucocytes | 4.2-9.4 | Tsnd/μL | 17.2 | 5.7 | 4.2 | X |
| Erythrocytes | 3.9-4.9 | Mio./μL | 5.15 | 4.37 | 4.2 | X |
| Hemoglobin | 11.3-15.1 | g/dl | 14.8 | 12.6 | 12.1 | |
| Hematocrit | 33-40 | % | 44.5 | 38.0 | 35.1 | X |
| MCV | 78-94 | fl | 86.4 | 87.0 | 83.6 | |
| MCH | 25-30 | pg | 28.7 | 28.8 | 28.8 | |
| MCHC | 32-35 | g/dl | 33.3 | 33.2 | 34.5 | |
| Thromocytes | 175-345 | Tsnd/μL | 147 | 316 | 226 | X |
| Lymphocytes | 18-50 | % | 34 | 55.2 | 44.5 | |
| Neutrophiles | 39-74 | % | 18.1 | 31.4 | 38.1 | |
| Basophiles | 0-1.5 | % | 1.1 | 1.1 | 1.4 | |
| Eosinophiles | 0-7.0 | % | 0.0 | 2.7 | 3.3 | |
| Monocytes | 4-12 | % | 3.2 | 9.6 | 12.7 | X |
| GOT/AST | <35 | U/I | 116 | 26 | 19 | X |
| (liver) | | | | | | |
| GPT (liver) | <35 | U/I | 285 | 42 | 15 | X |
| GGT (liver) | <33 | U/I | 123 | 47 | 18 | X |

Treatment of the infection with the analgesic Ibuprofen (against fever, pain and inflammation) in combination with Healthberry® 865 resulted in a decrease of the leukocytes as well as liver parameters back to normal or almost normal levels within 9 days after EBV diagnosis (see Table 6 day 9), which was further confirmed after 25 days including full physical recovery (e.g. participation in sports) after 18 days. Considering that EBV infections usually last minimum 3 weeks with time periods till full recovery lasting from additional weeks to months, this human case clearly displays the benefit of treating EBV infections with Healthberry® 865 as anti-viral and combining it with an analgesic in the therapy of these infections.

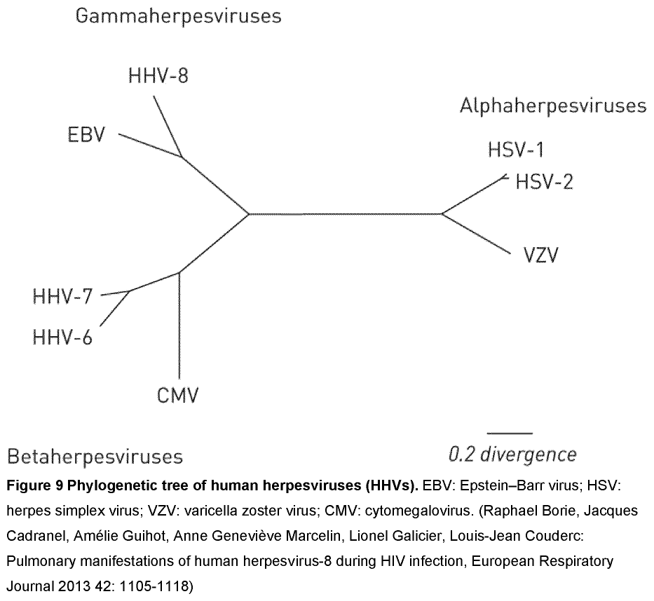

FIG. 9 shows the phylogenetic tree of human herpesviruses (HHVs). EBV: Epstein—Barr virus; HSV: herpes simplex virus; VZV: varicella zoster virus; CMV: cytomegalovirus. (Raphael Borie, Jacques Cadranel, Amélie Guihot, Anne Genevieve Marcelin, Lionel Galicier, Louis-Jean Couderc: Pulmonary manifestations of human herpesvirus-8 during HIV infection, European Respiratory Journal 2013 42: 1105-1118). It is obvious from the phylogenetic tree that the human herpesviruses, which were tested, are located at different arms of the phylogenetic tree, covering members of the Gammaherpesviruses, Alphaherpesviruses and Betaherpesviruses. Therefore, it is to be expected that the antiviral activity of the berry extracts covers the whole family of Herpesviridae.

1. A composition suitable for use in treating or preventing a virus infection in a subject, the composition comprising:
an extract of black currants and bilberries, wherein the virus is from the Herpesviridae family.

2. The composition of claim 1, wherein the black currants are a fruit of Ribes nigrum and/or the bilberries are a fruit of Vaccinium myrtillus, and/or
wherein the composition contains an extract from black currants and bilberries in a weight ratio of 0.5:1 to 1:0.5, and/or
wherein the extract is an extract of pomaces from the black currants and bilberries, and/or
wherein anthocyanins are present in the composition at a concentration of at least 25 weight-%, and/or
wherein the extract is an alcoholic extract, and/or
wherein the extract is prepared by a process comprising extracting black currants and/or bilberries to obtain first extract(s), purifying the first extract(s) via chromatography to obtain purified extract(s), mixing of the purified extract(s) with water to obtain a mixture, and spray-drying of the mixture.

3-6. (canceled)

7. The composition of claim 1, comprising:
cyanidin-3-glucoside, cyanidin-3-galactoside, cyanidin-3-arabinoside, delphinidin-3-glucoside, delphinidin-3-galactoside, delphinidin -3-arabinoside, petunidin-3-glucoside, petunidin-3-galactoside, petunidin-3-arabinose, peonidin-3-glucoside, peonidin-3-galactoside, peonidin-3-arabinose, malvidin-3-glucoside, malvidin-3-galactoside, malvidin-3-arabinose, cyanidin-3-rutinoside, and/or delphinidin-3-rutinoside.

8. The composition of claim 1, wherein the virus is herpes simplex virus-1 (HSV-1), herpes simplex virus-2 (HSV-2), Varicella zoster virus (VZV), Epstein—Barr virus (EBV), Cytomegalovirus (CMV), Roseolovirus, an/or or Kaposi's sarcoma-associated herpesvirus (KSHV, HHV-8), and/or
wherein the composition comprises anthocyanins and is suitable for administration to the subject in 1 to 10 oral dosages of at least 80 mg anthocyanins each per day, and/or
wherein the composition is configured to be administered to the subject, reaching a concentration in a target compartment of at least 30 μg/mL, and/or
wherein the composition is configured for use with a medical device which is suitable to be inserted into the subject, or the subject has had the medical device inserted, optionally transdermal or endotracheal.

9-11. (canceled)

12. The composition of claim 8, wherein the composition is suitable to be administered at a site of insertion of the medical device into the subject, and/or
wherein the medical device is configured for endotracheal intubation or parenteral nutrition, and/or
wherein the medical device is a needle, catheter, port, intubation device, tube, nebulizer, and/or implant, and/or
wherein a dwell time of the medical device in the subject is more than 24 hours.

13-15. (canceled)

16. The composition of claim 1, wherein the subject is a human, and/or
wherein the subject is a carrier of a Herpesviridae family virus, and/or
wherein the subject is infected with Kaposi's sarcoma-associated herpesvirus, and/or

17-19. (canceled)

20. The composition of claim 1, suitable for preventing or treating a cancer associated with a Herpesviridae family virus, optionally wherein:
the virus is EBV and the cancer is lymphoma (including Hodgkin lymphoma and Burkitts lymphoma), nasopharyngeal cancer, gastric cancer, or breast cancer; or
(ii) the virus is HHV-8 and the cancer is Kaposi's sarcoma, primary effusion lymphoma, HHV-8-associated multicentric Castleman disease, or breast cancer.

21. The composition of claim 1, suitable for preventing or treating an autoimmune disease associated with a Herpesviridae family virus, optionally wherein:
(i) the virus is EBV and the autoimmune disease is systemic lupus erythematosus (SLE), rheumatoid arthritis (RA), Sjögren's syndrome, or multiple sclerosis; or
(ii) the virus is HSV-1 and the autoimmune disease is multiple sclerosis.

22. The composition of claim 21, suitable for preventing or treating Alzheimer disease.

23. The composition of claim 22, which reduces β-amyloid plaque formation and/or brain tissue inflammation.

24. (canceled)

25. A topical composition, comprising:
an extract of black currants and bilberries; and
a pharmaceutically acceptable excipient suitable for a topical composition that is to be administered to skin.

26. An eye drop composition, comprising:
an extract of black currants and bilberries; and
a pharmaceutically acceptable excipient suitable for a composition that is to be administered to an eye.

27. A topical composition, comprising:
an analgesic; and
an extract of black currants and bilberries.

28. The composition of claim 25, comprising anthocyanins,
wherein the anthocyanins are present in the composition at a concentration of at least 25 weight-%.

29. A medical device suitable for insertion into a subject, the medical device comprising:
a coating composition on an exterior surface of the device,
wherein the coating composition comprises an extract of black currants and bilberries.

30. The medical device of claim 29, which is a needle, a catheter, a port, an intubation device, a tube, a nebulizer, and/or an implant.

31. A method of making the medical device of claim 29, the method comprising:
applying the coating composition to the exterior surface of the medical device, optionally wherein the coating composition is formulated as a cream, a hydrogel cream, or a spray.

32. A composition, comprising:
an antiviral agent; and
an extract of black currants and bilberries,
wherein the antiviral agent is a Herpesviridae antiviral agent.

33. The composition of claim 32, wherein the antiviral agent is acyclovir, ganciclovir, valganci clovir, foscarnet, famciclovir, penciclovir, valaciclovir or letermovir, and/or
wherein the composition is in topical composition form or eye drop form.

34. (canceled)

35. A method for preventing or reducing the risk of a virus infection in a cell or cells ex vivo, the method comprising:
contacting the cell or cells with a composition comprising an extract of black currants and bilberries,
optionally wherein the cell or cells are stem cells or CAR T cells,
optionally wherein the contacting comprises culturing or storing the cell or cells with the composition.
